(19) United States
(12) Patent Application Publication
(10) Pub. No.: US 2025/0009436 A1
(43) Pub. Date: Jan. 9, 2025

(54) MEDICAL INTERVENTION ARRANGEMENT AND COMPUTER-IMPLEMENTED METHOD FOR DETERMINING POSITION INFORMATION OF A MEDICAL INSTRUMENT

(71) Applicant: Siemens Healthineers AG, Forchheim (DE)

(72) Inventors: Stephan KELLNBERGER, Zorneding (DE); Oliver HORNUNG, Unterleinleiter (DE)

(73) Assignee: Siemens Healthineers AG, Forchheim (DE)

(21) Appl. No.: 18/761,381

(22) Filed: Jul. 2, 2024

(30) Foreign Application Priority Data
Jul. 5, 2023 (EP) 23183663.6

(51) Int. Cl. A61B 34/20; A61B 1/00; A61B 1/04; A61B 1/06; A61B 1/07; A61B 18/00; A61B 18/14

(52) U.S. Cl. CPC A61B 34/20; A61B 1/00009; A61B 1/042; A61B 1/0669; A61B 1/07; A61B 18/1477; A61B 2018/00577; A61B 2018/00982; A61B 2034/2055; A61B 2034/2065

(57) ABSTRACT

One or more example embodiments relates to a medical intervention arrangement, comprising an elongated medical instrument, such as a needle, for partial introduction into an intervention region of an examination object. In addition, one or more example embodiments relates to a computer-implemented method for determining position information of an elongated medical instrument, such as a needle, located in an intervention region of an examination object.

### CROSS-REFERENCE TO RELATED APPLICATION(S)

The present application claims priority under 35 U.S.C. § 119 to European Patent Application No. 23183663.6, filed Jul. 5, 2023, the entire contents of which is incorporated herein by reference.

### FIELD

One or more example embodiments relates to a medical intervention arrangement, comprising an elongated medical instrument, such as a needle, for partial introduction into an intervention region of an examination object. In addition, one or more example embodiments relates to a computer-implemented method for determining position information of an elongated medical instrument, such as a needle, located in an intervention region of an examination object.

### RELATED ART

In the prior art various types of minimally invasive interventions into an examination object, in particular a patient, have already been proposed. Minimally invasive interventions have the advantage of imposing as little stress as a possible on the patient. Here a medical instrument is introduced at least partly into an intervention region in a patient, wherein the medical instrument, in particular at the tip of the instrument, i.e. the distal end, usually has an intervention means, in particular an examination and/or treatment means. The medical instrument is pushed forwards into a destination position or into a destination region at which the intervention is to take place.

One example of this type of medical instrument are needles, for example biopsy needles, with which a tissue sample can be taken, and/or treatment data for treatment of the target tissue in the target region. Treatment needles here can in particular comprise ablation needles, with which diseased tissue, in particular tumor tissue, can be treated, in particular obliterated. For example energy emitters at the tip of the instrument have been proposed as treatment means, the output energy of which generates damage to tissue, for example by overheating of the tissue, and this destroys diseased tissue, for example tumors. Such interventions are known both for soft tissue, in particular in organs such as the liver, and also for musculoskeletal applications, for example spinal cord tumors.

In these minimally invasive interventions in particular however for the treatment of target tissue, it is extremely important to check the position of the medical instrument and to ensure that the target region or the target tissue is being reached. Various approaches have already been proposed for this in the prior art.

Firstly image-based monitoring of the positioning of the medical instrument is known. Computed tomography is usually employed as an imaging modality in this case, wherein in a “step-and-shoot” method a number of slice images are recorded during the insertion in order to carry out the monitoring. This does not allow monitoring in real time however, but rather a step-by-step process in which the person carrying out the intervention must leave room for each recording process. For specific types of interventions, in particular in the liver, it has also been proposed that ultrasound imaging be used for monitoring the positioning of the instrument, wherein for example tumors in liver parenchyma are not shown with a sufficiently high contrast and moreover, due to the deeper location with adipose patients, the image quality deteriorates. Bone structures also reduce the image quality here.

To support the person carrying out the intervention it has further been proposed that the path of the instrument be planned beforehand, for example with the aid a three-dimensional planning image dataset, and that an optical guidance facility, in particular a laser guidance facility, be used, wherein for example the medical instrument can be aligned in its longitudinal direction with the aid of a laser cross. The entry point can also be projected along with the angulation, in this case on the surface of the patient. However, even with such support, monitoring in respect of the path of forwards movement within the of the patient is still required, which with these types of system frequently occurs via imaging.

### SUMMARY

A reliable real-time monitoring during the forwards movement of medical instruments in the intervention region of the patient has so far proved difficult, so that real-time feedback, in particular with regard to the reaching of the target region and in an automated way, is not possible.

One or more example embodiments specifies a method for provision of feedback for forwards movement of a medical instrument in real time.

This may be achieved by an intervention arrangement with the features of claim 1 and by a method with the features of claim 15. Advantageous developments emerge from the dependent claims.

### BRIEF DESCRIPTION OF THE DRAWINGS

Advantages and details of one or more example embodiments are described below, as well as with the aid of the drawings. In which:

FIG. 1 shows a general functional schematic diagram of an inventive intervention arrangement,

FIG. 2 shows a diagram explaining interaction between light and material,

FIG. 3 shows the functional structure of a measuring device in a first embodiment variant,

FIG. 4 shows the functional structure of the measuring device in a second embodiment variant,

FIG. 5 shows a schematic diagram of a medical instrument in a first exemplary embodiment of the intervention arrangement,

FIG. 6 shows an example of a structure of an examination object,

FIG. 7 shows a graph of measuring data that is produced during advance through the structure of FIG. 6,

FIG. 8 shows a principle schematic diagram of a medical instrument in a second exemplary embodiment of the intervention arrangement,

FIG. 9 shows a detail of an optical waveguide in the region of a measuring position,

FIG. 10 shows a graph of the intensity of receive light with the advance for a number of measuring positions, and

FIG. 11 shows a flowchart of an exemplary embodiment of the inventive method.

### DETAILED DESCRIPTION

An inventive medical intervention arrangement has the following:
an elongated medical instrument, in particular a needle, for partial introduction into an intervention region of an examination object wherein, within the medical instrument at least one optical waveguide is routed to at least one distal measuring position, in which the optical waveguide has an opening for transmitting and receiving light, and
a measurement device arranged externally to the examination object, which is connected to the proximal end of the at least one optical waveguide and has:
a light source for creation of transmit light of a transmit spectrum to be guided by at least one of the at least one optical waveguides to the at least one measuring position,
a light detector for receiving receive light, which is guided from the at least one measuring position via at least one of the at least one optical waveguides to the measuring device, and
an evaluation unit for evaluation of the measuring data describing the receive spectrum of the receive light in the knowledge of the transmit spectrum for determining position information describing the position of the medical instrument in the examination object on the basis of reflection characteristics of materials, in particular tissues, of the examination object;
   and
a display facility for output of the position information.

In this case the position information is to be understood within the framework of the present invention not necessarily as an exact position specification but a position is for example also specified when a material class of the material at at least one measuring position, in particular a course of material classes along the distal portion of the medical instrument introduced into the examination object is known and/or will be determined whether or which measuring position or measuring positions are located within the examination object.

In accordance with one or more example embodiments it is thus proposed, via an optical waveguide, in particular at least one optical fiber, to use the reflection characteristics of tissue at the at least one measuring position in order to obtain the position information. In this case reflection characteristics not only describe the directed reflection, but also backscatter characteristics, i.e. the diffuse reflection. This corresponds to the usual division into transmission, absorption and reflection. Reflection characteristics can in such cases for example comprise a reflection coefficient and/or degree of reflection or can be described by these parameters.

The optical waveguide runs in this case inside and/or on the outside of the elongated medical instrument, in particular the needle. It is thus integrated and can be employed during the use of the medical instrument for determination of real-time information, here the position information. This real-time detection of the instrument course in the examination object for minimally invasive procedures is therefore provided.

Here, to particular advantage, a combination with laser guidance, generally a light guide facility that, based on planning, can display the entry point and the correct forwards movement orientation of the medical instrument, can be undertaken, since the inventive method of operation ultimately ideally supplements, namely in the support of the person carrying out the intervention adds position information still missing in respect of the forwards movement path. In particular there can therefore be provision for the intervention arrangement additionally to have a light guidance facility, in particular a laser guidance facility, for marking an entry point and/or a required orientation of the medical instrument in accordance with planning information. In this way the already outstanding support characteristics by the laser guidance are supplemented by a real-time detection of the (at least rough) forwards movement path.

In this case one or more example embodiments, generally speaking, is based on the idea that changes at the at least one measuring position, in particular on entry into the examination object, but preferably also for material borders within the examination object, give clear indications about the location of the corresponding measuring position. In concrete terms this can be used in various ways, which will be discussed below in even greater detail with respect to the concrete exemplary embodiments of the present invention. Actually the position information can preferably describe how far, at least approximately, the medical instrument is pushed into the examination object and/or in which kind of tissue/which tissue type or generally kind of material/material type the tip, i.e. the distal end of the instrument, is located.

In this way the use of x-ray imaging, in particular computed tomography imaging, is expediently greatly reduced, so that the examination object is subjected to lower x-ray doses. In this way, since the “step-and-shoot” method of operation is greatly reduced or becomes entirely unnecessary, the time needed for an intervention is also reduced. It is furthermore advantageous that the optical waveguide, in particular as at least one optical fiber, can easily be integrated into medical instruments, for example an ablation needle.

In general terms the intervention arrangement can also comprise a control facility as well, which can be built into the measuring device and/or be external to said device, and controls the overall operation of the intervention arrangement. For example the control facility can comprise an output unit for output of the position information at the display facility. If the intervention arrangement also comprises an imaging facility, this too, in particular in the context of the intervention, can be activated via a recording unit of the control facility, for example for recording the planning image dataset and/or for a control scan. The control facility can also have a planning unit for determination of planning information describing a planned instrument path. If a light guidance facility is also provided as part of the intervention arrangement, the control facility can also comprise an activation unit for activation of the light guidance facility for marking an entry point in accordance with the planning information and/or for marking an orientation of the medical instrument in accordance with the instrument path of the planning information. The activation unit can also activate further components.

The examination object can involve a male human or animal patient or a female human or animal patient and/or an intervention phantom. The instrument preferably involves a needle, in particular an ablation needle or other treatment needle, preferably for tumors. The medical instrument can however also involve another minimally invasive instrument, for example a catheter or the like.

In expedient exemplary embodiments there can be provision for the evaluation unit to be embodied, during the determination of the position information, to use a course of the measuring data arising over the advance in the examination object. If the course is considered, which in particular can describe various material class boundaries and/or changes in intensity, for example by additional measuring positions occurring in the examination object, along the advance, in general terms additional information is used, which allows a more precise and more robust determination of the position information. Concrete exemplary embodiments, which use the course of the measuring data, will be explained in even greater detail below.

In particular for exemplary embodiments in which conclusions are drawn about material classes from the reflection characteristics depicted by the measuring data, in particular tissue classes, various reflection behaviors of various materials play a role. Reflection characteristics are often wavelength-dependent, so that expedient embodiments of the present invention make provision for the transmit spectrum to cover a number of wavelengths and/or wavelength ranges. For example combinations of wavelengths and/or wavelength ranges can be chosen, which combinationally, with knowledge of the materials/material classes that arise, allow the desired distinctions to be made as well as possible. Thus for example oxygen-rich and oxygen-poor blood in the range around 700 nm has greatly different reflection characteristics. Examinations for optical characteristics of different materials, in particular of tissues, are to be found in the prior art and can also be used in the framework of the present invention.

Overall it can also be said that, in an advantageous way, not just one wavelength but a number of discrete wavelengths or wavelength ranges, in particular also a wide transmit spectrum, can be used in the transmit spectrum in order to allow a multispectral measurement. This further increases the accuracy and robustness of the measurement.

In general the spectrum can preferably lie in the visible or near-infrared wavelength range. At these frequencies suitable differences are evident in respect of the reflection characteristics, wherein the measurement is easy moreover for the patient to cope with.

While it basically conceivable, which will be discussed in greater detail for exemplary embodiments, to work independently of the respective materials, in particular tissue, an expedient development makes provision for the evaluation unit to be embodied to determine at least one material class, in particular tissue class, of the material reflecting the transmit light into the receive light at at least one of the at least one measuring position and/or a transition between such material classes, in particular tissue classes, as position information and/or with the determination of the position information from the measuring data. As already mentioned, the specification of a tissue class at which the at least one measuring position is located already represents expedient position information, for example when the measuring position coincides with a treatment means, since it can then be established whether the tissue is being reached. In other words there can be provision for the position information to describe an arrangement of at least one of the at least one measuring position in a section of material, in particular section of tissue, of the examination object. Yet the material class is also able to be used as an intermediate result on the way to position information, in particular when background knowledge for the intervention region is available that describes a location of sections of material, in a particular sections of tissue, of a material class in the intervention region in more concrete terms. If it is known from the background knowledge where specific material classes and boundaries of the material classes are located within the intervention region, through knowledge of the material classes with inclusion of the background knowledge, for example in the form of (possibly annotated) image data, at least portions of the position information can be obtained from this, for example also actual sections of materials, in particular sections of tissue, in which the measuring position and thus the instrument is located.

Use is made here of the fact that, as shown, the intensity reflected back can be correlated with different material classes, in particular tissue classes, in accordance with their reflection characteristics. If for example the coefficient of reflection of various material classes, in particular various tissue classes, as a function of the wavelength is known, with knowledge of the transmit spectrum it can be deduced from the receive spectrum which material or tissue is present at the measuring position. In other words a classification can be undertaken. For example for 750 nm oxygen carrying and non-oxygen carrying hemoglobin have different reflection characteristics, which allow a distinction. A similar behavior can be observed for other tissue classes, for example muscle tissue and fat tissue. Thus the measurement of the receive light and the assessment of the corresponding receive spectrum, in particular of wavelength-specific intensities, with knowledge of the transmit spectrum, allow a classification of the material at the location of the measuring position at which the receive light was reflected back.

In one or more example embodiments, there can be provision in this context for the evaluation unit to be embodied to take into account image data of the intervention region of the examination object in the classification and/or for assigning at least one of the at least one measuring positions to an or to the section of material visible in the image data. Sections of materials here are such sections that, during advance of the medical instrument, comprise a specific material class. In this connection it is especially expedient also to take into account the course of measuring data and thus the receive spectra with the advance, since the sections of material known from the image data on introduction of the medical instrument into the examination object is run through in the order shown in the image data. Here each transition between material classes, i.e. each corresponding e change in the receive spectrum, shows a section of change of material. If the image data is annotated or if the material class shown can be derived in another way, it can also be deduced solely from the image data and from the order of material classes in sections of materials produced from this, necessarily to be moved through, in which section of material of which material class the measuring position must be located after passing through a corresponding number of material transitions. In particular here, on determination of a material class from the receive spectrum, there can also be a plausibility check. Especially advantageously the image data also serves in any event however to allow a spatial assignment, since the spatial location of the sections of material in the image data is indeed known, in particular when the imaging facility with which it has been recorded is part of the intervention arrangement or at least a registration could take place. In exemplary embodiments it is thus conceivable to use material classes at a measuring position in order to determine the state of a corresponding measuring position in a section of material of this material class as position information, wherein, on entry into the section of material, the most accurate position determination is available.

In this case the image data can for example comprise a preliminary image dataset, in particular a planning image dataset, and/or image data of a control scan and/or of a live imaging. Preliminary image data and image data recorded during the intervention can or will expediently be registered with one another. In particular in the case of the preliminary image dataset, for example of the planning image dataset, the image data can also be annotated, as already briefly explained.

In a concrete example of a measuring position and of the ablation of a liver tumor, the measuring position, when the medical instrument is pushed into the examination object, in particular the patient, will pass the following material transitions or materials: skin, fat, blood, liver capsule, liver parenchyma and tumor. Whenever the receive spectrum changes it can be deduced that a new section of material has been entered, so that in particular with regard to the image data, the position of the medical instrument/of the measuring position can easily be traced at all times. A similar thing is also possible for musculoskeletal applications, when for example skin, fat and bone are passed before a tumor or other diseased tissue is reached.

Preferably with the use of image data the intervention arrangement, in particular as a part of the control facility, can further have an or the output unit, which is embodied for output of a display based on the image data, showing the position information. For example the section of material, in particular section of tissue, can be highlighted in the image data, in which the measuring position, for example at the tip of the instrument, or the instrument in general is currently located. In particular in combination with a light guidance facility, the planning information, which is based on a planning dataset, is used, this represents a simple and intuitive continuation of the support with respect to entry point and orientation, wherein in particular planning image data and additionally overlaid planning Information can be included as the basis of the display.

Preferably the evaluation unit can be embodied for the use of a trained function for classification. Thus pattern recognition/material classification, in particular tissue classification based on artificial intelligence, can also be employed in the framework of the present invention, wherein for training such a trained function for example, with the instrument at a measuring position arranged in a known material class, training measuring data or receive spectra can be recorded and used for learning the trained function. Here the training measuring data. also comprising in particular transitions between various material classes, can be recorded.

In general a trained function maps cognitive functions, which associate humans with other human brains. Through training based on training data (machine learning) the trained function is capable of adapting itself to new circumstances and of detecting and extrapolating patterns.

Generally speaking parameters of a trained function can be adapted by training. In particular supervised learning, semi-supervised learning, unsupervised learning, reinforcement Learning and/or active learning can be used. Above and beyond this representation learning (also known as “feature learning”) can be employed. The parameters of the trained function can in particular be adapted iteratively by a number of training steps.

A trained function can for example comprise a neural network, a Support Vector Machine (SVM), a decision tree and/or a Bayes network and/or the trained function can be based on k-means-clustering, Q-learning, genetic algorithms and/or assignment rules. In particular a neural network can be a deep neural network, a Convolutional Neural Network (CNN) or a deep CNN. Over and above this the neural network can be an Adversarial Network, a deep Adversarial Network and/or a Generative Adversarial Network (GAN).

As an alternative or in addition the parameters characterizing reflection, preferably wavelength-dependent, for example coefficient of reflections and the like, can also be determined by classical evaluation and compared with corresponding material properties, in particular tissue properties, for example in a database and/or a Look-Up table. Distinction criteria, as already explained above, can also be used in order to implement a classical evaluation.

In an advantageous concrete embodiment of the present invention there can be provision for at least one of the at least one measuring positions to be provided at a distal end of the instrument, in particular at an instrument tip. Treatment and/or examination means are mostly also at the distal end, in particular the tip of the instrument, so that particularly advantageously the arrangement allows at least one of the at least one measuring positions at the distal end to check in real time whether the target material to be treated and/or to be examined, in particular target tissue, i.e. the corresponding material class/tissue class, is being reached. Thus an increased reliability of the intervention is made possible. For a tumor ablation for example, before the beginning of the treatment a check can be made as to whether the distal end of the instrument and thus the treatment means is located inside tumor tissue. Via a target position arranged at the distant end it can thus be assessed especially easily whether the target region is being reached.

Expedient exemplary embodiments can however also provide for there to be provision for a number of measuring positions. While in this way it is possible more easily, with a number of treatment and/or examination means, to establish the actual surrounding material for each of these means or for other reasons, to establish material classes surrounding various sections of the medical instrument, an especially advantageous use of a number of measuring positions in respect of the determination of the position information, in particular also with respect to the advance path, is also conceivable.

In this context it is especially preferred for at least a part of the number of measuring positions to be provided spaced apart, in particular equidistantly, in the longitudinal direction along a distal portion of the medical instrument able to be introduced into the examination object. In this way, as the distal portion of the medical instrument is increasingly pushed into the examination object one after another, ever more measuring positions of materials of the examination object, in particular tissue, are covered, so that by contrast with measuring positions located outside of the examination object, reflections occur there, and the intensity rises. In this case, although it is basically conceivable, for each measuring position, to route separate optical waveguides for the transmit light and the receive light, it is however preferred when a common optical waveguide of the at least one optical waveguide is provided for guidance of the receive light from a number of measuring positions.

Then the evaluation unit can be embodied expediently,
to carry out the determination of the position information as a function of intensity of the receive light rising with the increasing number of measuring positions located within the examination object, and/or
to determine, for overall measuring data describing at least one measuring position (52) for receive light from a number of measuring positions (52), measuring position measuring data by subtraction of subtraction measuring data, in particular measured in isolation during a smaller advance and/or derived by subtraction during a smaller advance, from the overall measuring data.

If an instrument with a number of measuring positions known to the evaluation unit arranged along its longitudinal length is introduced into the examination object, the advance of the medical instrument can be measured by the light intensity of the overall receive light of all these measuring positions being evaluated. In general it can be said that due to the greater number of measuring positions that receive reflected receive light, a greater advance of the instrument into the examination object results in a higher intensity of the receive light. For example, the more measuring positions are covered by tissue, the back reflection and thus the intensity of the receive light increases. Whenever a new measuring position is pushed into the examination object, an increase occurs in the intensity of the receive light as a function of the reflection characteristics of the material of the examination object to a higher value, which can be correlated with the advance path of the medical instrument into the examination object as position information. In this way the position of the medical instrument in the examination object can be estimated.

Here the evaluation of the course via the advance is expedient, wherein, figuratively speaking, stages in the course that correspond to the number of measuring positions covered, which receive receive light reflected back, can be counted. Since it is known where the measuring positions lie, a path of advance range is produced. In particular a basic signal exists, which is also present when all measuring positions are located outside of the examination object. This increases in stages to new, higher average values of the intensity, when measuring positions are covered. As an alternative or in addition to a “counting” it is also conceivable however, through statistical evaluation and/or theoretical determination, to determine threshold values and/or ranges of threshold values, which each show how many measuring positions are located within the examination object for a measured intensity of the receive light.

The fact that the various measuring positions are introduced successively into the examination object, thus through each advancing measuring position, ultimately that which the subsequent measuring position will still measure is already measured, allows, by corresponding combination, for considered course, the receive spectrum to be determined, even with overlapping, i.e. measurement of receive light from a number of measuring positions, for individual measuring positions. If for example two measuring positions are located within the examination object, for a receive spectrum already measured for a smaller advance to the forward measuring position one can subtract from the overall receive spectrum in order to obtain the current measuring position receive spectrum for the forward measuring position and the like. This makes it possible, even with a number of measuring positions, which use a common optical waveguide for the receive light, to determine which material classes are present, as has been described above.

In this case it should be pointed out here that, for the use of a trained function for classification of material, it is also conceivable to train said function for overlapping of receive light of a number of measuring positions such that a separation and a determination of the corresponding material classes is also allowed, wherein then an assignment to measuring positions for example on grounds of background knowledge, in particular of image data, can take place.

In particular it is generally possible, if image data of the examination object is present, which in particular shows corresponding sections of materials, in particular sections of tissues, to reconcile overall receive spectra with conceivable combinations so that a more accurate determination of the location of the medical instrument in the examination object is possible than with the use of just one single measuring position, for example at the tip of the instrument. This improvement not only occurs for the combination with image data, but also already while taking into consideration previous measuring positions of material classes/sections of material already passed through. In general it should again be pointed out that naturally knowledge about the arrangement of the measuring positions on the medical instrument will be taken into consideration in the evaluation.

There can be provision for the optical waveguide to comprise at least one optical fiber. For example, a so-called “double-clad fiber” can be used, which usually has three optical layers, which have different refractive indices and in which the core and the first layer laid around it can both be used for light transport, here for example for transport of the transmit light and of the receive light via different “channels' of the double-clad fiber. Naturally other fibers and fiber structures can also be used as optical waveguides, for example a multimode fiber.

Basically it is conceivable that for at least one of the at least one measuring positions, different optical waveguides are provided for the guidance of the transmit light and of the receive light. It is also possible and preferred however that the measuring device when, for at least one of the at least one measuring positions, one of the at least one optical waveguides is provided both for guidance of the transmit light and also of the receive light, has an optical splitting unit for dividing up the receive light and the transmit light. Here for example usual beam splitters can be employed.

In the framework of the present invention the light detector can for example comprise a photon multiplier and/or a photon counter. Other embodiments basically known in the prior art are also conceivable.

The at least one of the at least one optical waveguides for guidance of transmit light to at least one of the at least one measuring positions can have an interruption point for coupling out of the transmit light at this measuring position. This is in particular expedient when an optical waveguide is used for a number of measuring positions, for example along the length of the medical instrument, since then, for example equidistant interruption points can be provided at the corresponding measuring positions in order to couple out transmit light in each case.

In concrete terms the optical waveguide, as already mentioned, can be routed integrated within the instrument, for example in a groove of an instrument shaft. It is also possible however to route the optical waveguide entirely inside the instrument and to provide windows through which light can pass at the measuring positions.

As already mentioned there can be provision for the instrument, in particular at its instrument tip, to have a treatment means for treatment of a target tissue class, in particular of tumor tissue, in particular the instrument is an ablation needle. In particular with respect to the destructive treatment of in particular diseased target tissue the present invention is especially expedient, since a check can be made as to whether the target region has also actually been reached.

The intervention arrangement can preferably further have an imaging facility with a patient couch for the examination object and, in particular, as part of the control facility, an activation unit, which is embodied for activation of the patient couch and/or of a recording arrangement of the imaging facility for positioning for a recording of image data with the imaging facility using the position information. This means that when a connection to the imaging facility and its positioning means is available, the position information, in particular in respect of the path of advance of the medical instrument, can also be used in order to set up the imaging facility correctly for a subsequent image recording, in particular so that the tip of the instrument is captured. In concrete terms it is expedient here to use the position information in order to bring the patient table and/or a recording arrangement of the imaging facility automatically into a target position for a control scan. This is in particular expedient with computed tomography facilities as imaging facilities when the intervention is not taking place in a single computed tomography slice, but here with increasing advance a change of the computed tomography slice is expedient. In this way a further expedient use of the position information facilitating the intervention is also provided.

As well as the intervention arrangement, one or more example embodiments also relates to a computer-implemented method for determination of position information of an elongated medical instrument located in an intervention region of an examination object, wherein at least one optical waveguide is routed within the medical instrument to least one distal measuring position, in which the optical waveguide has an opening for transmitting and receiving of light, and a measuring device arranged externally to the examination object, which is connected to the proximal end of the at least one optical waveguide, is used, having the following steps:
via a light source of the measuring device, creation of transmit light of a transmit spectrum by at least one of the at least one optical waveguides to be routed to the at least one measuring position,
via a light detector of the measuring device, receipt of receive light, which is routed from the at least one measuring position via at least one of the at least one optical waveguides to the measuring device,
via an evaluation unit of the measuring device, evaluation of measuring data describing the receive spectrum of the receive light in the knowledge of the transmit spectrum for determination of position information describing the position of the medical instrument in the examination object on the basis of reflection characteristics of materials, in particular tissues, of the examination object, and
output of the position information to a display facility.

All remarks regarding the inventive intervention arrangement can be transferred by analogy to the inventive method and vice versa, so that the advantages already stated can likewise be obtained with this.

Further conceivable is also a computer program, which has program means that, when the computer program is executed on a control facility of an intervention arrangement, causes said arrangement to carry out an inventive method. The computer program can be stored on an electronically readable data medium.

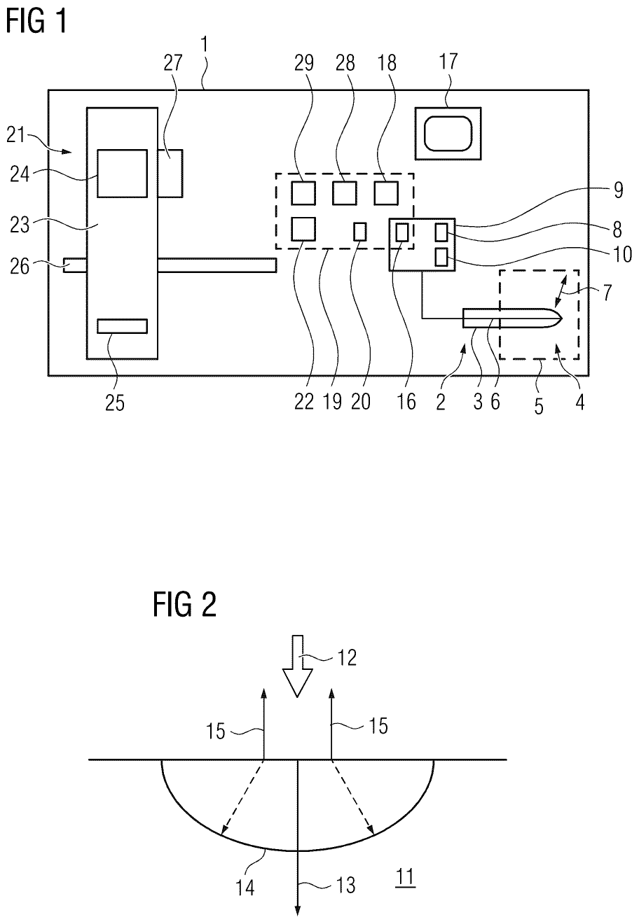

FIG. 1 shows a functional principle schematic diagram of an inventive intervention arrangement 1 for carrying out a minimally invasive intervention. The intervention arrangement 1, in the present example, comprises as a medical instrument 2 an elongated, at least essentially rigid, needle 3, for example an ablation needle for treatment of tumors, for example by heat, cold, high-frequency radiation or the like. To this end the needle 3 also has a treatment means not shown in FIG. 1 in greater detail for reasons of clarity.

The instrument 2 can be introduced with a distal portion, comprising the tip at the distal end 4, into an examination object 5 (not belonging to the intervention arrangement 1 and therefore merely indicated). Within the medical instrument 2 at least one optical waveguide 6 runs to at least one distal measuring position, thus provided on the distal portion, where transmit light can emerge from the optical waveguide 6 and if necessary receive light reflected by material present at the measuring position, in particular from the examination object 5, can enter the optical waveguide 6 again, cf. the double-ended arrow 7.

The transmit light is created via a light source 8, for example a laser, in a measuring device 9, with which the proximal end of the optical waveguide 6 is visibly connected. The measuring device further also comprises a light detector 10, with which the receive light can be detected. The light detector 10 can for example comprise a photo multiplier and/or a photon counter.

FIG. 2 shows schematically the interaction of light with a material 11, for example a tissue. The arrow 12 symbolizes incident transmit light. This transmit light is now partly transmitted (transmission, arrow 13), partly absorbed (absorption, symbolized by the curve 14) and partly reflected (reflection, cf. arrow 15). The reflection in this figure comprises both the directed reflection and also the diffuse reflection. The portion of reflected transmit light and thus the receive light is described by the reflection characteristics of the material 11, for example the coefficient of reflections.

If the transmit spectrum of the transmit light is known, which in the present example expediently comprises a number of wavelengths and/or wavelength ranges (multispectral measurement) and lies in the visible and/or near-infrared range, a distinction can be made between different materials 11 or different material classes are distinguished by observing the measuring data recorded by the light detector 10, which describes the receive spectrum, i.e. in particular intensities at various wavelengths. In a simple approach, which will likewise be discussed in even greater detail below, it can also be concluded merely through the increase of the intensity of the receive light, that now a reflection by material 11 is present that was not present previously.

To return to FIG. 1, an evaluation unit 16 of the measuring device 9 evaluates the measuring data with knowledge of the reflection characteristics and of the transmit spectrum in order to determine position information about the position of the medical instrument 2 in the examination object 5. In general terms the position information can for example describe a material class, in particular tissue class, within which is located at least one of the at least one measuring position, in exactly the same way as an advance path or an advance path area (as a rough position specification) and/or a section of material, in particular section of tissue, visible for example from image data, in which at least one of the at least one measuring positions or a specific portion of the medical instrument 2 is located. Concrete examples are presented below.

The evaluation unit 16 can use a trained function, as an alternative or in addition however, also distinguishing criteria, which are defined on the basis of known reflection behavior of expected materials of specific material classes, and/or a database or Look-Up Table, in which measuring data or receive spectra are assigned material classes, in order to deduce a material class for at least one of the at least one measuring positions. If a trained function is used, this can comprise a neural network, in particular a deep neural network, and be trained by virtue of training measuring data recorded from the known materials of the material classes. The determination of the material class, i.e. the classification, can be undertaken on the basis of the measuring data for receive light of a specific measuring position (measuring position measuring data), but also on the basis of overall measuring data describing overlaid receive light of a number of measuring positions, wherein it is also possible, on observation of the course of the measuring data over the path of advance into the examination object 5 by a combination consisting of overall measuring data, in particular by subtraction of subtraction measuring data, to deduce measuring position measuring data for individual measuring positions. Also in general it can be expedient for the evaluation unit 16 to use the course of the measuring data over the path of advance for the determination of the position information.

It is further expedient for background knowledge, in particular in the form of image data, to be included in the determination of the position information by the evaluation unit 16. Image data of the examination object 5, for example planning image data and/or also image data of a control scan, can already show sections of material, in particular sections of tissues that necessarily have to be traversed by the medical instrument 2 on advance into the examination object 5 and to which measuring positions can be assigned with the aid of the measuring data. Since, in the present example, the position information can also be output to a display facility 17, here to a monitor, of the intervention arrangement 1 in order to support the person carrying out the intervention, it can be expedient to create a presentation that shows position information, for example overlaid for the image data.

The evaluation unit 16 and also an output unit 18 activating the display facility 17 for output of the position information can also be part of a control facility 19 of the intervention arrangement 1 which, in the present example, is also arranged partly external to the measuring device 9. The control facility 19 comprises at least one processor and at least one memory means 20.

In the present exemplary embodiment the intervention arrangement 1 also comprises an imaging facility 21, which is able to be operated for recording at least one part of the image data already mentioned by a recording unit 22 of the control facility 19. The imaging facility 21, in the present example, involves a computed tomography facility, which has a gantry 23 with a recording arrangement guided therein, comprising an x-ray emitter 24 and an x-ray detector 25, as well as a patient couch 26 for placing of the examination object. Other imaging facilities are also conceivable, for example C-arm x-ray facilities.

Also arranged on the gantry 23 in the present example is a light guidance facility 27 of the intervention arrangement 1, in the present example a laser guidance facility. Since the control facility 19 in the present example also has a planning unit 28, via this, based on a planning image dataset, which is recorded by the imaging facility 21, planning information can be determined, which in the present example describes a planned instrument path in a target region to be treated, here a tumor, with target tissue to be treated, here tumor tissue, in such a way that a required entry point of the medical instrument 2, a required orientation of the medical instrument 2 and also a desired path of advance or the location of the target region per se are contained there or are able to be derived therefrom. This allows an activation unit 29 of the control facility 19 to activate the light guidance facility 27 in such a way that the required entry point can be marked on the surface of the examination object 5 and, for example via a laser cross, the orientation in accordance with the planning information can be predetermined. In addition to this support with respect to the orientation and the entry point, thanks to the measuring device 9 and the optical waveguide 6 and the determination and output of the position information made possible thereby, further outstanding support is provided for the person carrying out the intervention, who can now assess for example in the optimal manner whether the target region is reached as desired, or the medical instrument is suitably positioned for carrying out the treatment.

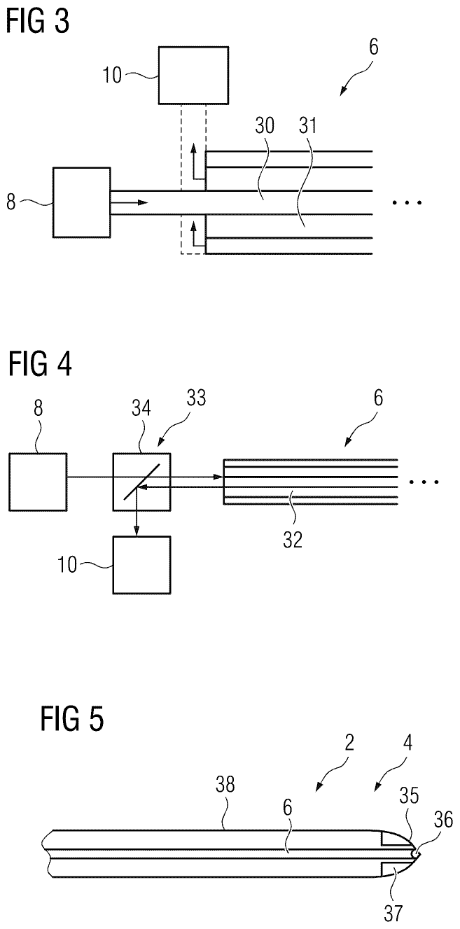

Various options for the optical waveguide 6 as well as the coupling in and coupling out of transmit light or receive light in the measuring device 9 exist within the framework of the present invention, of which two are explained by way of example in FIGS. 3 and 4. FIG. 3 shows an optical waveguide 6, which is embodied as a double-clad fiber. In this case, in the present example core 30 is used for transport of the transmit light starting from the light source 8, while the inner cladding 31 is employed for the return transport of the receive light, which then, as shown, is routed to the detector 10. Instead of a double clad optical fiber a multimodal fiber can also be employed; it is further conceivable to use two separate fibers or optical waveguides 6 for the transmit light and the receive light.

In the form of embodiment of FIG. 4 a usual, also single-clad optical fiber is shown as the optical waveguide 6, of which the core 32 is used both for transport of the transmit light from the light source 8 and also for transport of the receive light to the light detector 10, wherein a 50:50 bean splitter 34 is used in the present example as optical splitting unit 33 for splitting the transmit light and receive light.

FIG. 5 now shows a first concrete exemplary embodiment of a medical instrument 2 wherein, in the present example, for the sake of simplicity, only the distal portion with the distal end 4, i.e. the tip of the instrument 35, is shown. The in this case precisely one optical waveguide 6 is guided to a measuring position 36 at the tip of the instrument 35. Also provided at the tip of the instrument 35 in the present example is a treatment means 37, for example an ablation means. The optical waveguide 6 is routed in this example within the instrument shaft 38. The measuring position 36 can for example be formed by a window covering the end of the optical waveguide 6, but also directly by the end of the optical waveguide 6.

In such an embodiment it is especially expedient for the material class at the measuring position to be determined from the measuring data by the evaluation unit 36, as described above, since it can then in particular be checked whether the target tissue, i.e. the target region, is being reached. This can not only be undertaken with the aid of a classification by the trained function, the decision criteria and/or the database/Look-Up Table, but in addition or as an alternative also by inclusion of the expedient annotated image data in this case, since for example, over the course of the measuring data, transitions between different material classes, here tissue classes, can be counted. Preferable however is the classification, which in particular can be plausibility checked by the counting of the material transitions.

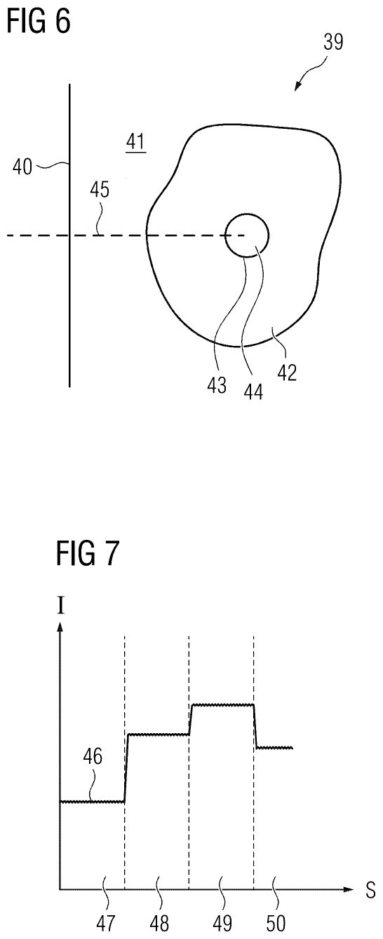

FIG. 6 shows schematically an intervention region 39 of the examination object 5 with the skin 40 as outer surface, fatty tissue 41 following on from it inwards, the transition to organ parenchyma 42 and also a tumor 43, which forms the target region, so that the tumor tissue 44 accordingly represents the target tissue. A planned instrument path 45 is likewise shown. The organ can involve a liver for example.

FIG. 7 shows purely schematically the graph 46 of a variable I able to be derived from the measuring data, allowing the distinction between the tissue classes fatty tissue, organ parenchyma and tumor tissue over the advance path S. Located in a region 47 is the measuring position 36 still outside of the examination object 5, here the patient, which is why only a basic signal is present. In region 48 the medical instrument 2, in concrete terms the measuring position 36, is guided through fatty tissue 41, which reflects the transmit light in accordance with the reflection characteristics of fatty tissue 41 partly in receive light and thus ensures another value of the variable. The transition into a region 49 (organ parenchyma 42) takes place, before in region 50, after a further transition once again to a new value of the variable the target tissue (tumor tissue 44) is reached.

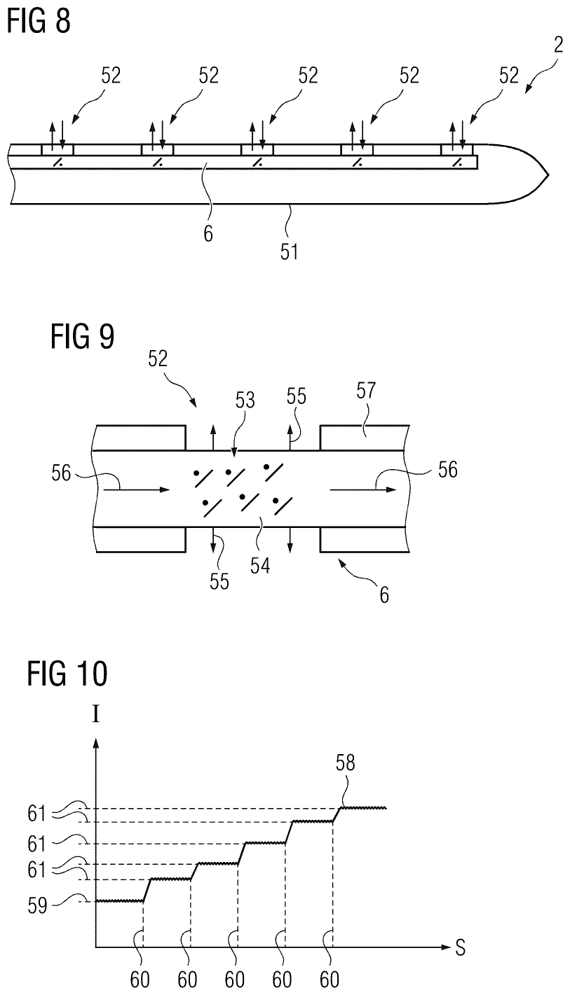

FIG. 8 illustrates a second concrete exemplary embodiment of a medical instrument 2, which in the present example has an optical waveguide 6 routed in a groove of the instrument shaft 51, to which a number of measuring positions 52 provided equidistantly along the length of the instrument 2 is assigned. This means that the optical waveguide 6 guides both the transmit light and also the receive light for all these measuring positions 52, wherein in the present example five measuring positions 52 are used for example, generally however another number, for example four to ten, can also be used. As is shown more precisely in FIG. 9, interruption points 53 in the optical waveguide 6, in concrete terms in the core 54 of the optical fibers, are provided at each measuring position 52, which couple out portions of the transmit light identified by arrows 55 through arrows 56. The cladding 57 of the optical fiber can be interrupted at these points.

In this second exemplary embodiment too the optical waveguide 6 can appropriately have openings at the measuring positions 52, but it is also possible to use windows.

In this second exemplary embodiment use is made of the fact that each measuring position 52 entering into the examination object 5 increases the intensity of the overall receive light, since, as from this point in time reflections occur from material, in particular tissue. This is illustrated by the characteristic intensity curve 58, is as shown purely schematically in FIG. 10. Once again the starting point in this case is a basic signal 59, for which all measuring positions 52 are arranged outside of the examination object 5, wherein with each advance point 60 a new measuring position 52 is covered by material, so that the intensity increases to a new average value 61. After five such stages all measuring positions 52 are located in the examination object 5. By counting the stages in curve 58 it can thus already be recognized how many measuring positions 52 are located in the examination object 5, from which as position information an advance path range can already be specified.

Especially preferably however material classes are determined here too, since naturally characteristic reflection characteristics underlying measuring data as well as material transitions can also be recognized in the curve 58. For this, as explained, measuring position measuring data can be extracted for individual measuring positions 52 or also an overall evaluation of the overall measuring data can be undertaken however. Here too a use of image data or a link to said data is expedient.

It should be pointed out that the exemplary embodiments of FIGS. 5 to 7 and 8 to 10 can naturally also be combined, for example the further measuring position 36 at the tip 35 as well as the optical waveguide 6 can be added to the measuring positions 52 and to the optical waveguide 6 connecting them.

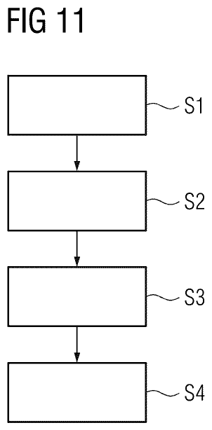

FIG. 11 finally shows a general flowchart of an exemplary embodiment of the inventive method, more precisely of its essential steps. In this what has been stated for the previous exemplary embodiments can naturally be transferred by analogy, for example as regards further steps and/or the practical implementation of steps.

In a step S1 transmit light is provided via the light source 8 and routed via the optical waveguide 6 to at least one of the at least one measuring positions 36, 52. Depending on the reflection characteristics of surrounding material, receive light reflected back now arises at the measuring position 36, 52, that is guided by the or by a further optical waveguide 6 to the light detector 10 that, in a step S2 accepts the measuring data that describes the receive spectrum of the receive light.

In a step S3 the measuring data is then evaluated in the evaluation unit 16 for the position information. The position information obtained in this way can be output in a step S4 on the display facility 17, in particular controlled by the output unit 18 and/or in a display based on the image data.

Although the invention has been illustrated and described in greater detail by one or more example embodiments, the invention is not restricted by the disclosed examples and other variations can be derived herefrom by the person skilled in the art, without departing from the scope of protection of the invention.

It will be understood that, although the terms first, second, etc. may be used herein to describe various elements, components, regions, layers, and/or sections, these elements, components, regions, layers, and/or sections, should not be limited by these terms. These terms are only used to distinguish one element from another. For example, a first element could be termed a second element, and, similarly, a second element could be termed a first element, without departing from the scope of example embodiments. As used herein, the term “and/or,” includes any and all combinations of one or more of the associated listed items. The phrase “at least one of” has the same meaning as “and/or”.

Spatially relative terms, such as “beneath,” “below,” “lower,” “under,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below,” “beneath,” or “under,” other elements or features would then be oriented “above” the other elements or features. Thus, the example terms “below” and “under” may encompass both an orientation of above and below. The device may be otherwise oriented (rotated 90 degrees or at other orientations) and the spatially relative descriptors used herein interpreted accordingly. In addition, when an element is referred to as being “between” two elements, the element may be the only element between the two elements, or one or more other intervening elements may be present.

Spatial and functional relationships between elements (for example, between modules) are described using various terms, including “on,” “connected,” “engaged,” “interfaced,” and “coupled.” Unless explicitly described as being “direct,” when a relationship between first and second elements is described in the disclosure, that relationship encompasses a direct relationship where no other intervening elements are present between the first and second elements, and also an indirect relationship where one or more intervening elements are present (either spatially or functionally) between the first and second elements. In contrast, when an element is referred to as being “directly” on, connected, engaged, interfaced, or coupled to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the “comprises,” terms “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list. Also, the term “example” is intended to refer to an example or illustration.

It should also be noted that in some alternative implementations, the functions/acts noted may occur out of the order noted in the figures. For example, two figures shown in succession may in fact be executed substantially concurrently or may sometimes be executed in reverse order, the depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

It is noted that some example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed above. Although discussed in a particular manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order. Although the flowcharts describe the operations as sequential processes, many of the operations may be performed in parallel, concurrently or simultaneously. In addition, the order of operations may be re-arranged. The processes may be terminated when their operations are completed, but may also have additional steps not included in the figure. The processes may correspond to methods, functions, procedures, subroutines, subprograms, etc.

Specific structural and functional details disclosed herein are merely representative for purposes of describing example embodiments. The present invention may, however, be embodied in many alternate forms and should not be construed as limited to only the embodiments set forth herein.

In addition, or alternative, to that discussed above, units and/or devices according to one or more example embodiments may be implemented using hardware, software, and/or a combination thereof. For example, hardware devices may be implemented using processing circuitry such as, but not limited to, a processor, Central Processing Unit (CPU), a controller, an arithmetic logic unit (ALU), a digital signal processor, a microcomputer, a field programmable gate array (FPGA), a System-on-Chip (SoC), a programmable logic unit, a microprocessor, or any other device capable of responding to and executing instructions in a defined manner. Portions of the example embodiments and corresponding detailed description may be presented in terms of software, or algorithms and symbolic representations of operation on data bits within a computer memory. These descriptions and representations are the ones by which those of ordinary skill in the art effectively convey the substance of their work to others of ordinary skill in the art. An algorithm, as the term is used here, and as it is used generally, is conceived to be a self-consistent sequence of steps leading to a desired result. The steps are those requiring physical manipulations of physical quantities. Usually, though not necessarily, these quantities take the form of optical, electrical, or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind that all of these and similar terms are to be associated with the appropriate physical quantities and are merely convenient labels applied to these quantities. Unless specifically stated otherwise, or as is apparent from the discussion, terms such as “processing” or “computing” or “calculating” or “determining” of “displaying” or the like, refer to the action and processes of a computer system, or similar electronic computing device/hardware, that manipulates and transforms data represented as physical, electronic quantities within the computer system's registers and memories into other data similarly represented as physical quantities within the computer system memories or registers or other such information storage, transmission or display devices.

In this application, including the definitions below, the term ‘module’ or the term ‘controller’ may be replaced with the term ‘circuit.’ The term ‘module’ may refer to, be part of, or include processor hardware (shared, dedicated, or group) that executes code and memory hardware (shared, dedicated, or group) that stores code executed by the processor hardware.

The module may include one or more interface circuits. In some examples, the interface circuits may include wired or wireless interfaces that are connected to a local area network (LAN), the Internet, a wide area network (WAN), or combinations thereof. The functionality of any given module of the present disclosure may be distributed among multiple modules that are connected via interface circuits. For example, multiple modules may allow load balancing. In a further example, a server (also known as remote, or cloud) module may accomplish some functionality on behalf of a client module.

Software may include a computer program, program code, instructions, or some combination thereof, for independently or collectively instructing or configuring a hardware device to operate as desired. The computer program and/or program code may include program or computer-readable instructions, software components, software modules, data files, data structures, and/or the like, capable of being implemented by one or more hardware devices, such as one or more of the hardware devices mentioned above. Examples of program code include both machine code produced by a compiler and higher level program code that is executed using an interpreter.

For example, when a hardware device is a computer processing device (e.g., a processor, Central Processing Unit (CPU), a controller, an arithmetic logic unit (ALU), a digital signal processor, a microcomputer, a microprocessor, etc.), the computer processing device may be configured to carry out program code by performing arithmetical, logical, and input/output operations, according to the program code. Once the program code is loaded into a computer processing device, the computer processing device may be programmed to perform the program code, thereby transforming the computer processing device into a special purpose computer processing device. In a more specific example, when the program code is loaded into a processor, the processor becomes programmed to perform the program code and operations corresponding thereto, thereby transforming the processor into a special purpose processor.

Software and/or data may be embodied permanently or temporarily in any type of machine, component, physical or virtual equipment, or computer storage medium or device, capable of providing instructions or data to, or being interpreted by, a hardware device. The software also may be distributed over network coupled computer systems so that the software is stored and executed in a distributed fashion. In particular, for example, software and data may be stored by one or more computer readable recording mediums, including the tangible or non-transitory computer-readable storage media discussed herein.

Even further, any of the disclosed methods may be embodied in the form of a program or software. The program or software may be stored on a non-transitory computer readable medium and is adapted to perform any one of the aforementioned methods when run on a computer device (a device including a processor). Thus, the non-transitory, tangible computer readable medium, is adapted to store information and is adapted to interact with a data processing facility or computer device to execute the program of any of the above mentioned embodiments and/or to perform the method of any of the above mentioned embodiments.

Example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed in more detail below. Although discussed in a particular manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order.

According to one or more example embodiments, computer processing devices may be described as including various functional units that perform various operations and/or functions to increase the clarity of the description. However, computer processing devices are not intended to be limited to these functional units. For example, in one or more example embodiments, the various operations and/or functions of the functional units may be performed by other ones of the functional units. Further, the computer processing devices may perform the operations and/or functions of the various functional units without sub-dividing the operations and/or functions of the computer processing units into these various functional units.

Units and/or devices according to one or more example embodiments may also include one or more storage devices. The one or more storage devices may be tangible or non-transitory computer-readable storage media, such as random access memory (RAM), read only memory (ROM), a permanent mass storage device (such as a disk drive), solid state (e.g., NAND flash) device, and/or any other like data storage mechanism capable of storing and recording data. The one or more storage devices may be configured to store computer programs, program code, instructions, or some combination thereof, for one or more operating systems and/or for implementing the example embodiments described herein. The computer programs, program code, instructions, or some combination thereof, may also be loaded from a separate computer readable storage medium into the one or more storage devices and/or one or more computer processing devices using a drive mechanism. Such separate computer readable storage medium may include a Universal Serial Bus (USB) flash drive, a memory stick, a Blu-ray/DVD/CD-ROM drive, a memory card, and/or other like computer readable storage media. The computer programs, program code, instructions, or some combination thereof, may be loaded into the one or more storage devices and/or the one or more computer processing devices from a remote data storage device via a network interface, rather than via a local computer readable storage medium. Additionally, the computer programs, program code, instructions, or some combination thereof, may be loaded into the one or more storage devices and/or the one or more processors from a remote computing system that is configured to transfer and/or distribute the computer programs, program code, instructions, or some combination thereof, over a network. The remote computing system may transfer and/or distribute the computer programs, program code, instructions, or some combination thereof, via a wired interface, an air interface, and/or any other like medium.

The one or more hardware devices, the one or more storage devices, and/or the computer programs, program code, instructions, or some combination thereof, may be specially designed and constructed for the purposes of the example embodiments, or they may be known devices that are altered and/or modified for the purposes of example embodiments.

A hardware device, such as a computer processing device, may run an operating system (OS) and one or more software applications that run on the OS. The computer processing device also may access, store, manipulate, process, and create data in response to execution of the software. For simplicity, one or more example embodiments may be exemplified as a computer processing device or processor; however, one skilled in the art will appreciate that a hardware device may include multiple processing elements or processors and multiple types of processing elements or processors. For example, a hardware device may include multiple processors or a processor and a controller. In addition, other processing configurations are possible, such as parallel processors.

The computer programs include processor-executable instructions that are stored on at least one non-transitory computer-readable medium (memory). The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc. As such, the one or more processors may be configured to execute the processor executable instructions.

The computer programs may include: (i) descriptive text to be parsed, such as HTML (hypertext markup language) or XML (extensible markup language), (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C#, Objective-C, Haskell, Go, SQL, R, Lisp, Java®, Fortran, Perl, Pascal, Curl, OCaml, Javascript®, HTML5, Ada, ASP (active server pages), PHP, Scala, Eiffel, Smalltalk, Erlang, Ruby, Flash®, Visual Basic®, Lua, and Python®.

Further, at least one example embodiment relates to the non-transitory computer-readable storage medium including electronically readable control information (processor executable instructions) stored thereon, configured in such that when the storage medium is used in a controller of a device, at least one embodiment of the method may be carried out.

The computer readable medium or storage medium may be a built-in medium installed inside a computer device main body or a removable medium arranged so that it can be separated from the computer device main body. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored for images, example, property information, may be stored in any other form, or it may be provided in other ways.

The term code, as used above, may software, firmware, and/or microcode, and may refer to programs, routines, functions, classes, data structures, and/or objects. Shared processor hardware encompasses a single microprocessor that executes some or all code from multiple modules. Group processor hardware encompasses a microprocessor that, in combination with additional microprocessors, executes some or all code from one or more modules. References to multiple microprocessors encompass multiple microprocessors on discrete dies, multiple microprocessors on a single die, multiple cores of a single microprocessor, multiple threads of a single microprocessor, or a combination of the above.

Shared memory hardware encompasses a single memory device that stores some or all code from multiple modules. Group memory hardware encompasses a memory device that, in combination with other memory devices, stores some or all code from one or more modules.

The term memory hardware is a subset of the term computer-readable medium. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding images, stored for example, property information, may be stored in any other form, or it may be provided in other ways.

The apparatuses and methods described in this application may be partially or fully implemented by a special purpose computer created by configuring a general purpose computer to execute one or more particular functions embodied in computer programs. The functional blocks and flowchart elements described above serve as software specifications, which can be translated into the computer programs by the routine work of a skilled technician or programmer.

Although described with reference to specific examples and drawings, modifications, additions and substitutions of example embodiments may be variously made according to the description by those of ordinary skill in the art. For example, the described techniques may be performed in an order different with that of the methods described, and/or components such as the described system, architecture, devices, circuit, and the like, may be connected or combined to be different from the above-described methods, or results may be appropriately achieved by other components or equivalents.

Independent of the grammatical term usage, individuals with male, female or other gender identities are included within the term.

1. A medical intervention arrangement, comprising:
an elongated medical instrument for partial introduction into an intervention region of an examination object, wherein within the elongated medical instrument at least one optical waveguide is routed to at least one distal measuring position, in which the optical waveguide has an opening for transmitting and receiving light; and
a measuring device external to the examination object, the measuring device being connected to a proximal end of the at least one optical waveguide, the measuring device including,
a light source configured to create transmit light of a transmit spectrum to be guided through at least one of the at least one optical waveguides to the at least one measuring position,
a light detector configured to receive receive light guided from the at least one measuring position via at least one of the at least one optical waveguides to the measuring device, and
an evaluation unit configured to evaluate measuring data describing a position a receive spectrum of the receive light based on of the transmit spectrum for determining position information describing the position of the elongated medical instrument in the examination object based on reflection characteristics of materials of the examination object; and
a display facility configured to output the position information.

2. The medical intervention arrangement of claim 1, wherein the evaluation unit configured to at least one use a course of the measuring data into the examination object to determine the position information, the transmit spectrum covering a number of wavelengths to determine the position information, or wavelength ranges to determine the position information.

3. The medical intervention arrangement of claim 1, wherein the evaluation unit is configured to at least one of,
determine at least one material class of a material at at least one of the at least one measuring position reflecting the transmit light into the receive light, or
determine a transition between material classes.

4. The medical intervention arrangement of claim 3, wherein the position information describes an arrangement at at least one of the at least one measuring position in a section of material of the examination object.

5. The medical intervention arrangement of claim 3, wherein the evaluation unit is configured to
use image data of the intervention region of the examination object to at least one of determine the at least one material class or assign at least one of the at least one measuring position to a section of material visible in the image data.

6. The medical intervention arrangement of claim 5, further comprising:
an output unit configured to output a display based on the image data showing the position information.

7. The medical intervention arrangement of claim 3, wherein the evaluation unit is configured to use a trained function to determine the at least one material class.

8. The medical intervention arrangement of claim 1, wherein at least one of the at least one measuring position is provided at a distal end of the elongated medical instrument.

9. The medical intervention arrangement of claim 1, wherein a number of measuring positions are provided.

10. The medical intervention arrangement of claim 9, wherein at least a part of the number of measuring positions are spaced apart in a longitudinal direction along a distal portion of the elongated medical instrument to be introduced into the examination object.

11. The medical intervention arrangement of claim 10, wherein
a common optical waveguide of the at least one optical waveguide is configured to guide the receive light from a number of measuring positions, and
the evaluation unit is configured to at least one of,
determine the position information as a function of an increasing intensity of the receive light as the number of measuring positions located in the examination object increases, or
for overall measuring data describing at least one measuring position for receive light from the number of measuring positions, determine measuring position measuring data by subtracting subtraction measuring data, from the overall measuring data.

12. The medical intervention arrangement of claim 1, wherein the at least one of the at least one optical waveguide for guidance of transmit light to at least one of the at least one measuring position has an interruption point for coupling out the transmit light at the at least one measuring position.

13. The medical intervention arrangement of claim 1, wherein the elongated medical instrument includes a treatment means at a tip for treatment of a target tissue class.

14. The medical intervention arrangement of claim 1, wherein further comprising:
an imaging facility including a patient couch for the examination object and an activation unit, the activation unit being configured to activate at least one of the patient couch or of a recording arrangement of the imaging facility for positioning for a recording of image data with the imaging facility using the position information.

15. A computer-implemented method for determination of position information of an elongated medical instrument located in an intervention region of an examination object wherein, within the elongated medical instrument at least one optical waveguide is guided to at least one distal measuring position, in which the optical waveguide has an opening for transmitting and receiving light, and a measuring device arranged externally to the examination object, which is connected to a proximal end of the at least one optical waveguide, is used, the method comprising:
creating, by a light source of the measuring device, transmit light of a transmit spectrum to be guided through at least one of the at least one optical waveguide to the at least one measuring position;
receiving, by a light detector of the measuring device, receive light, which is guided from the at least one measuring position via at least one of the at least one optical waveguide to the measuring device;
evaluating, by an evaluation unit of the measuring device, measuring data describing a receive spectrum of the receive light based on the transmit spectrum for determining position information describing the position of the elongated medical instrument in the examination object based on reflection characteristics of materials of the examination object; and
outputting the position information on a display facility.

16. The medical intervention arrangement of claim 1, wherein the reflection characteristics are reflection characteristics of tissues of the examination object.

17. The medical intervention arrangement of claim 8, wherein at least one of the at least one measuring position is at a tip of the instrument.

18. The medical intervention arrangement of claim 10, wherein the at least the part of the number of measuring positions are spaced apart equidistantly.

19. The medical intervention arrangement of claim 11, wherein the subtraction measuring data is at least one of measured in isolation during a smaller advance or derived by subtraction during a smaller advance.

20. The medical intervention arrangement of claim 13, wherein the treatment means is for treatment of a tumor tissue and the treatment means is an ablation needle.
